(19) United States
(12) Patent Application Publication
(10) Pub. No.: US 2021/0054451 A1
(43) Pub. Date: Feb. 25, 2021

(54) OPTIMIZING HIGH-THROUGHPUT SEQUENCING CAPACITY

(71) Applicant: Inscripta, Inc., Boulder, CO (US)

(72) Inventor: Kara Juneau, Boulder, CO (US)

(21) Appl. No.: 16/995,891

(22) Filed: Aug. 18, 2020

Related U.S. Application Data
(60) Provisional application No. 62/888,945, filed on Aug. 19, 2019.

(51) Int. Cl. C12Q 1/6874; C40B 20/00

(52) U.S. Cl. CPC C12Q 1/6874; C40B 20/00

(57) ABSTRACT

Provided are methods and compositions for preparing nucleic acid fragments for sequencing by synthesis on a flow cell. The methods and compositions described herein introduce nucleotide diversity into a sample preparation that would otherwise lack nucleotide diversity due to homogeneity of the sequencing target.

### RELATED CASES

The present application claims priority to U.S. Ser. No. 62/888,945, filed 19 Aug. 2019.

### FIELD OF THE INVENTION

This invention relates to methods and compositions of matter for optimizing sample library preparation and throughput capacity for massively parallel next-generation nucleic acid sequencing platforms.

### BACKGROUND OF THE INVENTION

In recent years, advances in next-generation sequencing (NGS) platforms have resulted in dramatically increased throughput capacity and significantly reduced sequencing costs. This progress has allowed for rapid and affordable resequencing of large numbers of genomes; de novo genome sequencing of many diverse microbial, plant, and animal species; targeted resequencing studies, e.g., exome sequencing; and increasingly sensitive variant detection in human disease contexts such as cancer.

One NGS method, reversible terminator sequencing by synthesis (SBS), relies on fluorescent detection of the sequential addition of modified dNTPs complementary to a template fragment, i.e., the sample to be sequenced, immobilized on a solid support in the context of a flow cell. Millions of synthesis reactions proceed in parallel through a cycle of nucleotide incorporation, fluorescent imaging, and cleavage, yielding hundreds of megabases to gigabases of sequencing read data per run. During these first ˜25 cycles, analysis software is trained to recognize the fluorescent signal specific to each nucleotide, and phasing/pre-phasing, color matrix corrections, and pass filter calculations also occur. These training and correction steps are critical to accurate base calling and quality score calculations; however, nucleotide diversity within the template library is critical to the training and correction steps.

One solution for technical challenges resulting from low nucleotide diversity in, e.g., samples of homogenous template fragments, is to spike-in a whole-genome sample with high nucleotide diversity to ensure that all four bases are represented in every cycle. The PhiX genomic library—derived from the bacteriophage PhiX genome—is commonly used for this purpose. A spike-in of 5-50% PhiX genomic library may be required depending on the homogeneity of the sequencing sample. As a result, 5-50% of reads will correspond to PhiX DNA, decreasing the reads corresponding to the target sample by 5-50%.

There is thus a need in the art of SBS sample preparation for improved methods and compositions for introducing nucleotide diversity to address the necessary training and correction steps associated with SBS without decreasing useful reads corresponding to the target sample rather than devoting sequencing capacity to unproductive control sequences. The present disclosure addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

Provided herein are compositions of matter and methods for introducing nucleotide diversity to one or more sequencing samples that inherently lack nucleotide diversity due to having identical or nearly identical nucleotide bases at their ends. For SBS applications, low nucleotide diversity of the template sequencing sample results in poor quality data. Homogenous nucleic acid template ends result in biased base composition in each SBS cycle and prevents data analysis software from accurately identifying DNA clusters and performing accurate base calling. Thus, in instances of SBS where the sequencing template fragments are identical or nearly identical and inherently lack nucleotide diversity, heterogeneity must be introduced by some other means. Compared to previous solutions to this problem, the methods and compositions disclosed herein introduce nucleotide diversity to the sample using “shifty primers” or “shifty oligonucleotide primers” without sacrificing sequencing capacity or necessitating the pooling of four or more unique samples in a sequencing run.

The present disclosure provides in one embodiment a “two-step” configuration for preparing samples for SBS, where the first step employs forward-priming and backward-priming oligonucleotides, referred to herein as SP1 shifty primers, comprising: user-designed homology regions directed to amplify a nucleic acid template; a phase-shift region of variable length which may include 1, 2, 3, 4, 5, 6, 7, 8, 9, or more nucleotides (a “shifty sequence”); a primer binding site for subsequent amplification by additional oligonucleotide primers disclosed herein; a target sequence for annealing by a first SBS primer; a target sequence for annealing by a second SBS primer; a target sequence for annealing by a first indexing primer; or a target sequence for annealing by a second indexing primer. The second step employs two additional forward-priming and backward-priming oligonucleotides, referred to herein as SP2 primers, which include regions configured to bind to the primer binding sites of the SP1 shifty primers described above, user-defined indexing sequences for differentiating between multiplexed samples, and adapter sequences P5 and P7 that mediate annealing to the oligonucleotides bonded to the solid support surface of the flow cell.

In an alternative embodiment, the present disclosure provides a “one-step” configuration where the forward-priming and backward-priming shifty oligos comprise: user-designed homology regions directed to amplify a nucleic acid template; a phase-shift region of variable length which may include 1, 2, 3, 4, 5, 6, 7, 8, 9, or more nucleotides (the “shifty sequence”); user-defined indexing sequences for differentiating between multiplexed samples; adapter sequences P5 and P7 that mediate annealing to the oligonucleotides bonded to the solid support surface of the flow cell; a target sequence for annealing of a first SBS primer; a target sequence for annealing of a second SBS primer; a target sequence for annealing by a first indexing primer; and a target sequence for annealing of a second indexing primer. After amplification with the shifty oligonucleotides provided herein according to the methods disclosed herein, the target sequencing sample fragment will comprise all the required sequence features and nucleotide diversity at its ends to yield high-quality SBS data.

The present disclosure also provides methods employing the SP1 and SP2 oligonucleotide primers described above to prepare nucleic acid fragments for sequencing by synthesis on a flow cell. In particular, the methods disclosed herein are useful for introducing nucleotide diversity to a sample preparation that would otherwise lack nucleotide diversity due to homogeneity of the sequencing target.

During amplification of a target DNA sequence with the shifty oligonucleotide primers disclosed herein, the reaction mixture includes a heterogeneous mix of SP1 shifty primers comprising phase-shift sequences of varying length. In the resulting amplicons, therefore, there is varying distance or shift between the target sequence for annealing of the SBS primer and the first nucleotide of target sequence, from molecule to molecule. When sequencing by synthesis proceeds from the target sequence for annealing of the SBS primer within the adapter sequence, each cycle of nucleotide incorporation includes sufficient nucleotide diversity for successful cluster identification, color training, and base-calling.

Amplification of a target DNA sequence with the oligonucleotide primers disclosed herein accomplishes the dual purposes of adding the necessary adapter sequences, including all the sequence features required for SBS on a flow cell, to the ends of the DNA fragments while also introducing nucleotide diversity to what would otherwise be a homogenous sequencing sample via a “phase shift” of nucleotides (e.g., a “shifty sequence” component of a “shifty primter”). As described above, homogenous sequencing samples present problems to sequencing by synthesis, namely cluster identification, color training, and accurate base calling.

Thus, one embodiment presents a set of oligonucleotides configured to introduce nucleotide diversity into sample DNA to be sequenced comprising: a first primer comprising from 5′ to 3′, a first SP2 binding site, a first variable-length phase-shift sequence n nucleotides in length, and a region homologous to a 5′ region of the sample DNA; a second primer comprising from 5′ to 3′, the first SP2 binding site, a second variable-length phase-shift sequence n+1 nucleotides in length, and the region homologous to a 5′ region of the sample DNA; a third primer comprising from 5′ to 3′, the first SP2 binding site, a third variable-length phase-shift sequence n+2 nucleotides in length, and the region homologous to a 5′ region of the sample DNA; a fourth primer comprising from 5′ to 3′, the first SP2 binding site, a fourth variable-length phase-shift sequence n+3 nucleotides in length, and the region homologous to a 5′ region the sample DNA; a fifth primer comprising from 5′ to 3′, a second SP2 binding site, a fifth variable-length phase-shift sequence n nucleotides in length, and a region homologous to a 3′ region of the sample DNA; a sixth primer comprising from 5′ to 3′, the second SP2 binding site, a sixth variable-length phase-shift sequence n+1 nucleotides in length, and the region homologous to a 3′ region of the sample DNA; a seventh primer comprising from 5′ to 3′, the second SP2 binding site, a seventh variable-length phase-shift sequence n+2 nucleotides in length, and the region homologous to a 3′ region of the sample DNA; and an eighth primer comprising from 5′ to 3′, the second SP2 binding site, an eighth variable-length phase-shift sequence n+3 nucleotides in length, and the region homologous to a 3′ region of the sample DNA; wherein the first, second, third and fourth primers have different nucleotides at a 5′ position of their variable-length phase-shift sequences, and wherein the fifth, sixth, seventh and eighth primers have different nucleotides at a 5′ position of their variable-length phase-shift sequences.

In some aspects of this 8-primer embodiment, except for the 5′ position, the first, second, third, fourth, fifth, sixth, seventh and eighth variable-length phase-shift sequences are a same sequence of nucleotides and in alternative aspects, the first, second, third, fourth, fifth, sixth, seventh and eighth variable-length phase-shift sequences are different sequences.

In some aspects of the 8-primer embodiment, the first, second, third and fourth primers further comprise: a first SP1 binding site configured to hybridize to the first SP2 binding site, an indexing sequence, and a P5 sequence if fifth, sixth, seventh, and eighth primers comprise a P7 sequence and the P7 sequence if the fifth, sixth, seventh, and eighth primers comprise the P5 sequence; and the fifth, sixth, seventh, and eighth primers further comprise: a second SP1 binding site configured to hybridize to the second SP2 binding site, an indexing sequence, and the P7 sequence if the first, second, third and fourth primers comprise the P5 sequence and the P5 sequence if the first, second, third and fourth primers comprise the P7 sequence. In some aspects, the P5 sequence is 5′-AATGATACGGCGACCACCCA-3′ [SEQ ID NO. 16] and the P7 sequence is 5′-CAAGCAGAAGACGGCATACGAGAT-3′ [SEQ ID NO. 17].

In other aspects of the 8-primer embodiment, the set of oligonucleotides further comprises a ninth primer comprising: a first SP1 binding site configured to hybridize to the first SP2 binding site, an indexing sequence, and a P5 sequence if a tenth primer comprises a P7 sequence and the P7 sequence if the tenth primer comprises the P5 sequence; and the tenth primer comprising: a second SP1 binding site configured to hybridize to the second SP2 binding site, an indexing sequence, and the P7 sequence if the ninth primer comprises the P5 sequence and the P5 sequence if the ninth primer comprises the P7 sequence.

In some aspects of the 8-primer embodiment, the first, second, third, fourth, fifth, sixth, seventh, and eighth primers are represented at about equimolar ratios.

In some aspects of the 8-primer embodiment, the first, second, third, fourth, fifth, sixth, seventh, and eighth variable-length phase-shift sequences have a sequence motif A.

In alternative aspects of the 8-primer embodiment, the set of oligonucleotides further comprises a ninth primer comprising from 5′ to 3′, the first SP2 binding site, a ninth variable-length phase-shift sequence n nucleotides in length, and the region homologous to a 5′ region of the sample DNA; a tenth primer comprising from 5′ to 3′, the first SP2 binding site, a tenth variable-length phase-shift sequence n+1 nucleotides in length, and the region homologous to a 5′ region of the sample DNA; an eleventh primer comprising from 5′ to 3′, the first SP2 binding site, a eleventh variable-length phase-shift sequence n+2 nucleotides in length, and the region homologous to a 5′ region of the sample DNA; a twelfth primer comprising from 5′ to 3′, the first SP2 binding site, a twelfth variable-length phase-shift sequence n+3 nucleotides in length, and the region homologous to a 5′ region the sample DNA; a thirteenth primer comprising from 5′ to 3′, a second SP2 binding site, a thirteenth variable-length phase-shift sequence n nucleotides in length, and a region homologous to a 3′ region of the sample DNA; a fourteenth primer comprising from 5′ to 3′, the second SP2 binding site, a fourteenth variable-length phase-shift sequence n+1 nucleotides in length, and the region homologous to a 3′ region of the sample DNA; a fifteenth primer comprising from 5′ to 3′ the second SP2 binding site, a fifteenth variable-length phase-shift sequence n+2 nucleotides in length, and the region homologous to a 3′ region of the sample DNA; and a sixteenth primer comprising from 5′ to 3′, the second SP2 binding site, a sixteenth variable-length phase-shift sequence n+3 nucleotides in length, and the region homologous to a 3′ region of the sample DNA; wherein the ninth, tenth, eleventh and twelfth primers have different nucleotides at a 5′ position of their variable-length phase-shift sequences, wherein the thirteenth, fourteenth, fifteenth and sixteenth primers have different nucleotides at a 5′ position of their variable-length phase-shift sequences, and wherein the variable-length phase-shift sequence of each of the ninth, tenth, eleventh and twelfth primers is a different sequence than the variable-length phase-shift sequence of the first, second, third, fourth, fifth, sixth, seventh and eighth primers. In some aspects, the ninth, tenth, eleventh and twelfth variable-length phase-shift sequences are different sequences than the thirteenth, fourteenth, fifteenth and sixteenth variable-length phase-shift sequences.

In some aspects of the 16-primer embodiment, the first, second, third and fourth primers further comprise: a first SP1 binding site configured to hybridize to the first SP2 binding site, an indexing sequence, and a P5 sequence if the fifth, sixth, seventh, and eighth primers comprise a P7 sequence and the P7 sequence if the fifth, sixth, seventh, and eighth primers comprise the P5 sequence; the fifth, sixth, seventh, and eighth primers further comprise: a second SP1 binding site configured to hybridize to the second SP2 binding site, an indexing sequence, and the P7 sequence if the first, second, third and fourth primers comprise the P5 sequence and the P5 sequence if the first, second, third and fourth primers comprise the P7 sequence; the ninth, tenth, eleventh and twelfth primers further comprise: the first SP1 binding site configured to hybridize to the first SP2 binding site, an indexing sequence, and the P5 sequence if the thirteenth, fourteenth, fifteenth and sixteenth primers comprise the P7 sequence and the P7 sequence if the thirteenth, fourteenth, fifteenth and sixteenth primers comprise the P5 sequence; and the thirteenth, fourteenth, fifteenth and sixteenth primers further comprise: the second SP1 binding site configured to hybridize to the second SP2 binding site, an indexing sequence, and the P7 sequence if the ninth, tenth, eleventh and twelfth primers comprise the P5 sequence and the P5 sequence if the ninth, tenth, eleventh and twelfth primers comprise the P7 sequence.

In other aspects of the 16-primer embodiment, except for the 5′ position, the first, second, third, fourth, fifth, sixth, seventh and eight variable-length phase-shift sequences have a sequence motif A and the ninth, tenth, eleventh, twelfth, thirteenth, fourteenth, fifteenth and sixteenth variable-length phase-shift sequences have a sequence motif B, and wherein sequence motif A and sequence motif B are different.

In alternative aspects of the 16-primer embodiment, the set of shifty oligonucleotide primers further comprises a seventeenth primer comprising: a first SP1 binding site configured to hybridize to the first SP2 binding site, an indexing sequence, and a P5 sequence if a tenth primer comprises a P7 sequence and the P7 sequence if the tenth primer comprises the P5 sequence; and an eighteenth primer comprising: a second SP1 binding site configured to hybridize to the second SP2 binding site, an indexing sequence, and the P7 sequence if the ninth primer comprises the P5 sequence and the P5 sequence if the ninth primer comprises the P7 sequence.

In yet other aspects of the 16-primer embodiment, the set of oligonucleotides further comprises a seventeenth primer comprising from 5′ to 3′, the first SP2 binding site, a seventeenth variable-length phase-shift sequence n nucleotides in length, and the region homologous to a 5′ region of the sample DNA; an eighteenth primer comprising from 5′ to 3′, the first SP2 binding site, an eighteenth variable-length phase-shift sequence n+1 nucleotides in length, and the region homologous to a 5′ region of the sample DNA; a nineteenth primer comprising from 5′ to 3′, the first SP2 binding site, a nineteenth variable-length phase-shift sequence n+2 nucleotides in length, and the region homologous to a 5′ region of the sample DNA; a twentieth primer comprising from 5′ to 3′, the first SP2 binding site, a twentieth variable-length phase-shift sequence n+3 nucleotides in length, and the region homologous to a 5′ region the sample DNA; a twenty-first primer comprising from 5′ to 3′, a second SP2 binding site, a twenty-first variable-length phase-shift sequence n nucleotides in length, and a region homologous to a 3′ region of the sample DNA; a twenty-second primer comprising from 5′ to 3′, the second SP2 binding site, a twenty-second variable-length phase-shift sequence n+1 nucleotides in length, and the region homologous to a 3′ region of the sample DNA; a twenty-third primer comprising from 5′ to 3′ the second SP2 binding site, a twenty-third variable-length phase-shift sequence n+2 nucleotides in length, and the region homologous to a 3′ region of the sample DNA; and a twenty-fourth primer comprising from 5′ to 3′, the second SP2 binding site, a twenty-fourth variable-length phase-shift sequence n+3 nucleotides in length, and the region homologous to a 3′ region of the sample DNA; wherein the seventeenth, eighteenth, nineteenth and twentieth primers have different nucleotides at a 5′ position of their variable-length phase-shift sequences, wherein the twenty-first, twenty-second, twenty-third and twenty-fourth primers have different nucleotides at a 5′ position of their variable-length phase-shift sequences, and wherein the seventeenth, eighteenth, nineteenth, twentieth, twenty-first, twenty-second, twenty-third and twenty-fourth variable-length phase-shift sequences are different sequences than the first, second, third, fourth, fifth, sixth, seventh, eighth, ninth, tenth, eleventh, twelfth, thirteenth, fourteenth, fifteenth and sixteenth variable-length phase-shift sequences.

In some aspects of the 24-primer embodiment, except for the 5′ position, the first, second, third, fourth, fifth, sixth, seventh and eighth variable-length phase-shift sequences have a sequence motif A, the ninth, tenth, eleventh, twelfth, thirteenth, fourteenth, fifteenth and sixteenth variable-length phase-shift sequences have a sequence motif B, and the seventeenth, eighteenth, nineteenth, twentieth, twenty-first, twenty-second, twenty-third and twenty-fourth variable-length phase-shift sequences have a sequence motif C, and wherein sequence motif A, sequence motif B and sequence motif C are different.

Other embodiments provide a set of oligonucleotides configured to introduce nucleotide diversity into sample DNA to be sequenced comprising: a first primer comprising from 5′ to 3′, a P5 or P7 sequence, an indexing sequence, a first SP2 binding site, a first variable-length phase-shift sequence n nucleotides in length, and a region homologous to a 5′ region of the sample DNA; a second primer comprising from 5′ to 3′, the P5 or P7 sequence, the indexing sequence, the first SP2 binding site, a second variable-length phase-shift sequence n+1 nucleotides in length, and the region homologous to a 5′ region of the sample DNA; a third primer comprising from 5′ to 3′, the P5 or P7 sequence, the indexing sequence, the first SP2 binding site, a third variable-length phase-shift sequence n+2 nucleotides in length, and the region homologous to a 5′ region of the sample DNA; a fourth primer comprising from 5′ to 3′, the P5 or P7 sequence, the indexing sequence, the first SP2 binding site, a fourth variable-length phase-shift sequence n+3 nucleotides in length, and the region homologous to a 5′ region the sample DNA; a fifth primer comprising from 5′ to 3′, the P5 or P7 sequence, the indexing sequence, a second SP2 binding site, a fifth variable-length phase-shift sequence n nucleotides in length, and a region homologous to a 3′ region of the sample DNA; a sixth primer comprising from 5′ to 3′, the P5 or P7 sequence, the indexing sequence, the second SP2 binding site, a sixth variable-length phase-shift sequence n+1 nucleotides in length, and the region homologous to a 3′ region of the sample DNA; a seventh primer comprising from 5′ to 3′, the P5 or P7 sequence, the indexing sequence, the second SP2 binding site, a seventh variable-length phase-shift sequence n+2 nucleotides in length, and the region homologous to a 3′ region of the sample DNA; and an eighth primer comprising from 5′ to 3′, the P5 or P7 sequence, the indexing sequence, the second SP2 binding site, an eighth variable-length phase-shift sequence n+3 nucleotides in length, and the region homologous to a 3′ region of the sample DNA; wherein the first, second, third and fourth primers have different nucleotides at a 5′ position of their variable-length phase-shift sequences, and wherein the fifth, sixth, seventh and eighth primers have different nucleotides at a 5′ position of their variable-length phase-shift sequences and wherein if the first, second, third and fourth primers comprise the P5 sequences, the sixth, seventh, eighth and ninth primers comprise the P7 sequences and wherein if the first, second, third and fourth primers comprise the P7 sequences, the sixth, seventh, eighth and ninth primers comprise the P5 sequences.

In some aspects of this embodiment, except for the 5′ position, the first, second, third, fourth, fifth, sixth, seventh and eighth variable-length phase-shift sequences are a same sequence of nucleotides. In other aspects of this embodiment, the first, second, third, fourth, fifth, sixth, seventh and eighth variable-length phase-shift sequences are different sequences.

Yet another embodiment presents a set of oligonucleotides configured to introduce nucleotide diversity into sample DNA to be sequenced comprising: a first primer comprising from 5′ to 3′, a first SP2 binding site, a first variable-length phase-shift sequence n nucleotides in length, and a region homologous to a 5′ region of the sample DNA; a second primer comprising from 5′ to 3′, the first SP2 binding site, a second variable-length phase-shift sequence n+1 nucleotides in length, and the region homologous to a 5′ region of the sample DNA; a third primer comprising from 5′ to 3′, the first SP2 binding site, a third variable-length phase-shift sequence n+2 nucleotides in length, and the region homologous to a 5′ region of the sample DNA; a fourth primer comprising from 5′ to 3′, the first SP2 binding site, a fourth variable-length phase-shift sequence n+3 nucleotides in length, and the region homologous to a 5′ region the sample DNA; a fifth primer comprising from 5′ to 3′, a first SP2 binding site, a fifth variable-length phase-shift sequence n+4 nucleotides in length, and a region homologous to a 5′ region of the sample DNA; a sixth primer comprising from 5′ to 3′, the first SP2 binding site, a sixth variable-length phase-shift sequence n+5 nucleotides in length, and the region homologous to a 5′ region of the sample DNA; a seventh primer comprising from 5′ to 3′, the first SP2 binding site, a seventh variable-length phase-shift sequence n+6 nucleotides in length, and the region homologous to a 5′ region of the sample DNA; an eighth primer comprising from 5′ to 3′, the first SP2 binding site, a eighth variable-length phase-shift sequence n+7 nucleotides in length, and the region homologous to a 5′ region the sample DNA; a ninth primer comprising from 5′ to 3′, a second SP2 binding site, a ninth variable-length phase-shift sequence n nucleotides in length, and a region homologous to a 3′ region of the sample DNA; a tenth primer comprising from 5′ to 3′, the second SP2 binding site, a tenth variable-length phase-shift sequence n+1 nucleotides in length, and the region homologous to a 3′ region of the sample DNA; an eleventh primer comprising from 5′ to 3′ the second SP2 binding site, an eleventh variable-length phase-shift sequence n+2 nucleotides in length, and the region homologous to a 3′ region of the sample DNA; a twelfth primer comprising from 5′ to 3′, the second SP2 binding site, a twelfth variable-length phase-shift sequence n+3 nucleotides in length, and the region homologous to a 3′ region of the sample DNA; a thirteenth primer comprising from 5′ to 3′, the second SP2 binding site, a thirteenth variable-length phase-shift sequence n+4 nucleotides in length, and a region homologous to a 3′ region of the sample DNA; a fourteenth primer comprising from 5′ to 3′, the second SP2 binding site, a fourteenth variable-length phase-shift sequence n+5 nucleotides in length, and the region homologous to a 3′ region of the sample DNA; a fifteenth primer comprising from 5′ to 3′ the second SP2 binding site, a fifteenth variable-length phase-shift sequence n+6 nucleotides in length, and the region homologous to a 3′ region of the sample DNA; and a sixteenth primer comprising from 5′ to 3′, the second SP2 binding site, a sixteenth variable-length phase-shift sequence n+7 nucleotides in length, and the region homologous to a 3′ region; wherein the first, second, third, fourth, fifth, sixth, seventh and eighth primers have different nucleotides at a 5′ position of their variable-length phase-shift sequences, and wherein the ninth, tenth, eleventh, twelfth, thirteenth, fourteenth, fifteenth and sixteenth primers have different nucleotides at a 5′ position of their variable-length phase-shift sequences.

Other embodiments provide a method for introducing nucleotide diversity into one or more sequencing samples, the method comprising providing a fragment of DNA to be sequenced; providing a set of “shifty” oligonucleotide primers; and amplifying the fragment of DNA by PCT using the shifty oligonucleotides.

These aspects and other features and advantages of the invention are described below in more detail.

### BRIEF DESCRIPTION OF THE FIGURES

FIGS. 1A, 1B, and 1C are three schematics depicting an exemplary target amplicon, a pair of SP1 shifty oligonucleotide primers, a pair of SP2 oligonucleotide primers, and a final PCR product that is the template for subsequent sequencing by synthesis and indexing. The three schematics illustrate successive rounds of amplification, first by SP1 shifty primers to introduce the phase-shift regions and SBS sequencing primer binding sites, second by SP2 primers to introduce unique indexing sequences, and the final amplification product which is the target for sequencing by synthesis on the flow cell.

FIGS. 2A and 2B are two schematics depicting an exemplary target amplicon, a pair of shifty oligonucleotide primers, and a final PCR product that is the template for subsequent sequencing by synthesis and indexing. The two schematics represent PCR amplification to introduce the phase-shift regions; SBS sequencing primer binding sites, unique indexing sequences, and yield a final amplification product which is the target for sequencing by synthesis on the flow cell.

FIG. 3A shows the nucleotide sequences of an ILLUMINA™ sequencing primer 5′ of an exemplary set of four shifty sequences. FIG. 3B shows an exemplary set of shifty primers where the set comprises sixteen different shifty sequences and sixteen different lengths. FIG. 3C shows an exemplary set of shifty primers where the set comprises sixteen different shifty sequences having four different lengths. FIG. 3D shows an exemplary set of shifty primers where the set comprises four different shifty sequences (four motifs) and four lengths. (Note, FIGS. 3A-3D show shifty sequences and shifty primers for forward primers only.)

### DETAILED DESCRIPTION

All of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry, biological emulsion generation, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; Berg et al. (2002) Biochemistry, 5^{th} Ed., W.H. Freeman Pub., New York, N.Y.; all of which are herein incorporated in their entirety by reference for all purposes. CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of ordinary skill in the art that the present invention may be practiced without one or more of these specific details. In other instances, well-known features and procedures well known to those skilled in the art have not been described in order to avoid obscuring the invention.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TTAGCTGG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

“Homology” or “identity” or “similarity” refers to sequence similarity between two peptides or, more often in the context of the present disclosure, between two nucleic acid molecules. The term “homologous region” or “homology arm” refers to a region on the donor DNA with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not be located on the same contiguous DNA molecule (i.e. chromosome) and may still have interactions resulting in altered regulation.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. Promoters may be constitutive or inducible. A “pol II promoter” is a regulatory sequence that is bound by RNA polymerase II to catalyze the transcription of DNA.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2a; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The terms “target genomic DNA sequence”, “target sequence”, or “genomic target locus” refer to any locus in vitro or in vivo, or in a nucleic acid (e.g., genome or episome) of a cell or population of cells, in which sequencing is desired. The target sequence can be a genomic locus or extrachromosomal locus.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like.

The present disclosure describes methods and compositions for introducing nucleotide diversity into one or more sequencing samples that may inherently lack nucleotide diversity due to having identical or nearly identical ends. Reversible terminator sequencing by synthesis (SBS) relies on fluorescent detection of the sequential addition of modified dNTPs complementary to a template fragment, i.e., the sample to be sequenced, immobilized on a solid support. Millions of synthesis reactions proceed in parallel through a cycle of nucleotide incorporation, fluorescent imaging, and cleavage, yielding hundreds of megabases to gigabases of sequencing read data per run. These parallel reactions take place in a flow cell, which is a plate a glass comprising several lanes, the surfaces of which are covered in an array of oligonucleotides covalently bonded to the glass surface. There may be two species of oligos coating the glass surface, each complementary to adapters ligated to one or the other end of the template nucleic acid to be sequenced. The SBS workflow from sample to data interpretations includes sample preparation, cluster generation on the solid support, sequencing by synthesis, and software-based data analysis.

Sample preparation typically involves ligating adapters to the ends of the DNA fragments to be sequenced. The adapters may include barcoding or indexing sequences, sequencing primer binding sites, regions complementary to the oligos that are covalently bonded to the solid surface of the flow cell, among other features. Target nucleic acids may also be amplified by PCR prior to ligating adaptor sequences.

During cluster generation, nucleic acid fragments anneal to the oligonucleotides coating the surface of the flow cell, mediated by complementarity between one of the two species of oligonucleotides and the ligated adapter sequence. The second adapter sequence anneals to the second species of oligonucleotides coating the surface of the flow cell and fragments are clonally amplified by repeated cycles of bridge amplification. One end of each bridge is liberated from the flow-cell surface resulting in single stranded templates for the first sequencing read.

Sequencing by synthesis begins with the sequencing primer annealing to the adapter sequence of the free end of the immobilized template fragment. A polymerase proceeds by incorporating fluorescently tagged nucleotides that include a reversible terminator. Only one nucleotide is incorporated in each round, determined by the sequence of the template. Based on unique emission from each of the four bases, incorporation of each nucleotide is detected and read. Terminators are removed and the next nucleotide is incorporated, detected, and read. This cycle is repeated for the desired read length. This sequencing by synthesis process is repeated for the reverse strand to generate pair-end reads in the forward and reverse directions.

During the first four to seven cycles of SBS, images of fluorescent signals emitted from nucleotide incorporation are used by analysis software to locate each cluster in the flow cell in a process called template generation. During the first 25 cycles or so, analysis software is also trained to recognize the fluorescent signal specific to each nucleotide and phasing/pre-phasing, color matrix corrections, and pass filter calculations occur. These training and correction steps are critical to accurate base calling and quality score calculations.

Nucleotide diversity within the template library is important for these training and correction steps during early cycles of SBS. Diverse and balanced libraries contain all four bases at similar percentages for each cycle and allow for accurate template generation and color-training. However, for homogenous samples where the ends of each fragment are identical and nucleotide diversity is extremely low or zero—such as amplicon-based library preparations—template generation and color-training may fail leading to low-quality data and many fewer reads per sequencing run.

One solution to the technical challenges presented is to spike-in a whole-genome sample with high nucleotide diversity to ensure that all four bases are represented in every cycle. The PhiX genomic library, derived from the bacteriophage PhiX genome, is commonly used for this purpose. Depending on the homogeneity of the sequencing sample, a spike-in of 5-50% PhiX genomic library may be required. As a result, 5-50% of reads will correspond to PhiX DNA, decreasing the useful reads corresponding to the target sample by 5-50%.

One prior art solution to the problem with lack of nucleotide diversity was to ligate specialized sequencing adapters to nucleic acid fragments that have homogenous ends. The adapters are of four different lengths, i.e. n, n+1, n+2, n+3, thereby staggering the base of the homogenous end read during a particular SBS cycle. This approach requires pooling at least four separate samples in an SBS run, where each sequencing sample receives a unique adapter length. In isolation each single unique adapter does not solve the low nucleotide diversity problem; however when four or more samples are pooled, each sample has a different adapter length and the first base of the homogenous ends will be read during different cycles, thereby conferring high nucleotide diversity to each cycle.

A major disadvantage to this staggered-length adapter approach is that at least four samples need to be pooled very carefully to ensure that all four adapter lengths are represented in the SBS run. If less than four samples are pooled, then effective staggering is not achieved resulting in the nucleotide diversity problem and accompanying low-quality data.

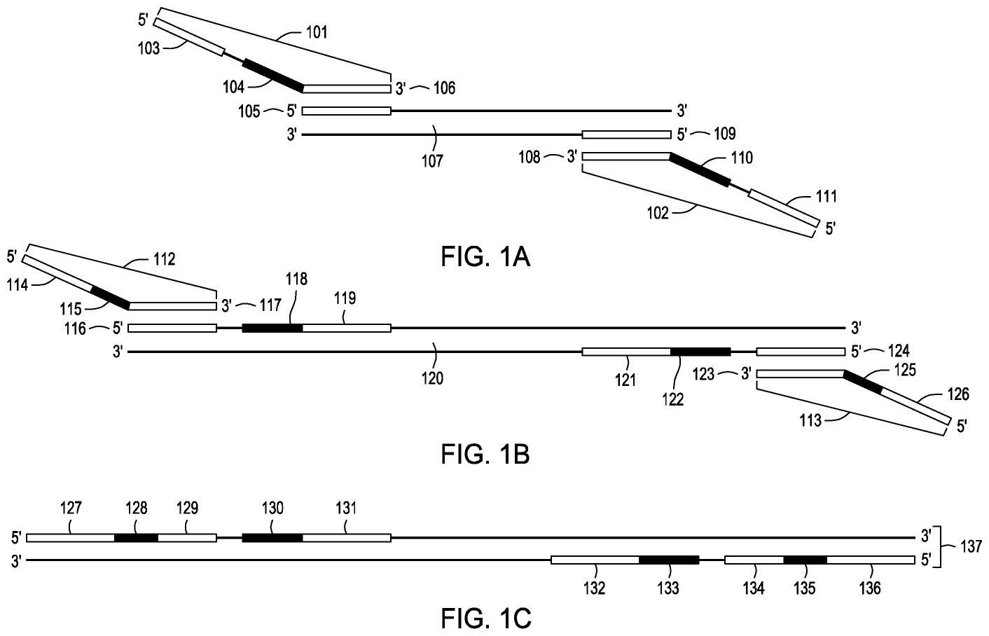

The methods and compositions disclosed herein include PCR reactions with user-designed “shifty” oligonucleotide primers to prepare target DNA sequence for SBS applications. In one embodiment—a “two-step” embodiment—a first sequencing PCR reaction referred to as the SP1 “shifty” reaction, schematized in FIG. 1A, and a second sequencing PCR reaction referred to as the SP2 reaction, schematized in FIG. 1B, are performed. The final product of these two PCR reactions, the target DNA fragment that is prepared for SBS on a flow cell, is represented in FIG. 1C. User-defined target DNA 107 is the template for the SP1 reaction and SP1 reaction products 120 are the template for the SP2 reaction. User-designed oligonucleotide primers are used in these reactions, with SP1 primers 101, 102 directed to target DNA 107 in the SP1 reaction, and SP2 primers 112, 113 directed to SP1 reaction products 120 in the SP2 reaction.

SP1 primers 101, 102 contain three key features: target DNA binding sites 106, 108, phase-shift sequences 104, 110, and SP2 primer binding sites 103, 111. The target DNA binding sites 106, 108 include the 3′ terminal end of the SP1 primers 101, 102. The target DNA binding sites 106, 108 are user-defined and designed to be complementary to regions 105, 109 flanking the target DNA 107 that is to be amplified and prepared for downstream SBS. An SP1 forward primer 101 and SP1 reverse primer 102 will together amplify the intervening sequence 107 between their binding sites 105, 109 in the SP1 PCR reaction of FIG. 1A. The target DNA binding sites may be between 10 and 100 nucleotides in length.

The phase-shift sequences 104, 110 are 5′ adjacent to the target DNA binding sites 106, 108 and may be 1, 2, 3, 4, 5, 6, 7, 8, 9, or more nucleotides in length. An important feature of the present disclosure is that the SP1 reaction is performed with an equimolar mix of eight distinct SP1 oligonucleotide primers: four forward and four reverse oligonucleotide primer species. For example, the SP1 forward primers 101 may include phase-shift regions 104 of 6, 7, 8, and 9 nucleotides in length. The four SP1 reverse primers 102 may also include phase-shift regions 110 of 6, 7, 8, and 9 nucleotides in length. The four species of SP1 forward primers 101 are identical to each other except for the phase-shift region 104, and the four species of SP1 reverse primers 102 are identical to each other except for the phase-shift region 110. Because of the equimolar mix of primer species in the SP1 reaction of FIG. 1A, the products of the SP1 reaction will be of incrementally varying lengths according to the particular SP1 primer pairs 101, 102 that amplified any particular amplicon.

The SP2 primer binding sites 103, 111 within the SP1 primers 101, 102 are positioned at the 5′ terminal ends of the oligonucleotide primers. The SP2 primer binding sites 103, 111 are user-defined to be targeted by the SP2 primers 112, 113 in the SP2 reaction of FIG. 1B. The SP2 primer binding sites 103, 111 may be between 10 and 100 nucleotides in length.

SP2 primers 112, 113 contain three key features: SP1 amplicon target binding sites 117, 123, indexing sequences (i.e. sample tag or barcode) 115, 125, and P5 or P7 sequences 114, 126 for annealing to oligonucleotides immobilized on the surface of the flow cell. P5 and P7 priming sequences are well-known primers for sequencing by synthesis, where P5 is 5′-AATGATACGGCGACCACCCA-3′ [SEQ ID NO. 16] and 5′-CAAGCAGAAGACGGCATACGAGAT-3′ [SEQ ID NO. 17].

The SP1 amplicon target binding sites 117, 123 include the 3′ terminal end of the SP2 oligonucleotide primers 112, 113. The SP1 amplicon target binding sites 117, 123 are user-defined and designed to be complementary to the SP2 primer binding sites 116, 124 that flank the amplicon products 120 of the SP1 reaction of FIG. 1A, including phase-shift region 118, target DNA binding sites 119, target DNA binding site 121, phase-shift region 122. The SP2 forward primer 112 and SP2 reverse primer 113 together amplify in the SP2 reaction of FIG. 1B the amplicon products 120 of the SP1 reaction which are located between the SP2 forward primer binding site 116 and reverse primer binding site 124. The SP1 amplicon target binding site may be between 10 and 100 nucleotides in length.

The indexing sequences (i.e. sample tag or barcode) 115, 125 are unique user-defined identifying sequences that may be used to identify and distinguish samples during pooled SBS. Unique indexing sequences are assigned to each sample in the pool and resulting reads can be sorted and assigned to the sample from which they were read. Indexing sequences may be between 5 and 50 nucleotides in length.

The P5 and P7 sequences 114, 126 are positioned at the 5′ terminal ends of the SP2 oligonucleotide primers 112, 113. The P5 sequence 114 typically is located at the 5′ terminal end of the forward SP2 primer 112 and the P7 sequence 126 typically is located at the 5′ terminal end of the reverse SP2 primer 113. The P5 and P7 sequences 114, 126 allow the library fragment 137 to attach to a flow cell surface by annealing to complementary oligos immobilized to the glass. Before attachment to the flow cell, the target DNA fragments 137 are denatured, and thus a single-stranded copy of the target DNA is sequenced.

The amplicon products 137 of the SP2 reaction of FIG. 1B, illustrated in FIG. 1C, include the cumulative features of the SP1 reaction of FIG. 1A and the SP2 reaction of FIG. 1B, namely: P5 sequence 127, indexing sequence (i.e. sample tag or barcode) 128, SP1 amplicon target binding sites 129, phase-shift region 130, target DNA binding sites 131, target DNA binding site 132, phase-shift region 133, SP2 primer binding site 134, indexing sequence (i.e. sample tag or barcode) 135, and P7 sequence 136. Amplicon products 137 may be purified and used as input material for downstream SBS applications. Phase-shift regions 130, 133 introduce nucleotide diversity to what would otherwise have been homogenous DNA ends with identical or nearly identical nucleotide sequence.

The target DNA 107 for amplification by SP1 phase-shift primers 101, 102 may be DNA amplicons generated by PCR, and may be amplified from any number of DNA or cDNA sources including plasmids, BACs, YACs, cosmids, phosmids, genomic DNA, cDNA reverse transcribed from total RNA extracts, cDNA reverse transcribed from ribosomal RNA, synthetic oligos, PCR products, and the like.

Alternatively, the target DNA 107 for amplification by SP1 phase-shift primers 101, 102 may be a circular plasmid. SP1 phase-shift primers are designed to amplify any segment of the circular plasmid DNA and capture the intervening sequence, resulting in amplicons flanked by phase-shift adapters described above. The target DNA for amplification by the SP1 phase-shift primers also may be a circular plasmid that has been edited by a nucleic acid-guided nuclease system. SP1 phase-shift primers are designed to flank the region of the circular plasmid DNA that has been edited and capture the edit and surrounding sequence, resulting in amplicons flanked by phase-shift adapters described above.

Alternatively, the target DNA 107 for amplification by SP1 phase-shift primers 101, 102 may be commercially synthesized DNA fragments. SP1 phase-shift primers are designed to amplify any segment of the commercially synthesized DNA fragments and capture the intervening sequence, resulting in amplicons flanked by phase-shift adapters described above. The target DNA for amplification by the SP1 phase-shift primers also may be commercially synthesized DNA fragments that have been edited by a nucleic acid-guided nuclease system. SP1 phase-shift primers are designed to flank the region of the commercially synthesized DNA that has been edited and capture the edit and surrounding sequence, resulting in amplicons flanked by phase-shift adapters described above.

Additionally, the target DNA 107 for amplification by SP1 phase-shift primers 101, 102 may be a linear or circular DNA molecule that is the product of isothermal assembly. It may be desirable to sequence the junctions of the fragments that comprise the assembled mixture, in which case a user may design SP1 phase-shift primers to flank the junctions and sequence across them. The junctions are thus captured by amplification with SP1 phase-shift primers, resulting in amplicons flanked by phase-shift adapters described above.

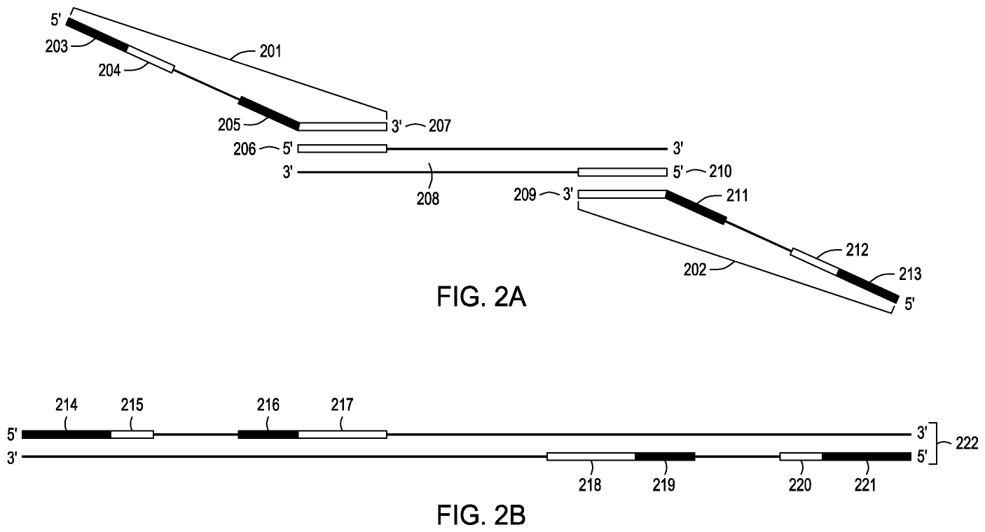

In an alternative embodiment, the sequence features of the SP1 primer pairs and the SP2 primer pairs may be combined such that a single PCR reaction is performed with a single pair of oligonucleotide primers, schematized in FIG. 2A. Oligonucleotide primer 201 combines the sequence features of forward SP1 primer 101 and forward SP2 primer 112, and oligonucleotide primer 202 combines the sequence features of reverse SP1 primer 102 and reverse SP2 primer 113. A single PCR reaction can be performed to yield amplicon 222, the target DNA fragment that is prepared for SBS on a flow cell, represented in FIG. 2B.

User-defined target DNA 208 is the template for PCR reaction with user-designed oligonucleotide primers 201 and 202. Primers 201 and 202 contain four key features: target DNA binding sites 207, 209, phase-shift sequences 205, 211, indexing sequences (i.e. sample tag or barcode) 204, 212, and P5 or P7 sequences 203, 213 for annealing to oligonucleotides immobilized on the surface of the flow cell.

The target DNA binding sites 207, 209 are located at the 3′ terminal end of the PCR primers 201, 202. The target DNA binding sites 207, 209 are user-defined and designed to be complementary to regions 206, 210 flanking the target DNA 208 that is to be amplified and prepared for downstream SBS. Forward primer 201 and reverse primer 202 will together amplify the intervening sequence 207 between binding sites 206, 210 in the PCR reaction of FIG. 2A. The target DNA binding sites 206, 210 may be between 10 and 100 nucleotides in length.

The phase-shift sequences 205, 211 are located 5′ of and adjacent to the target DNA binding sites 207, 209 and may be 1, 2, 3, 4, 5, 6, 7, 8, 9, or more nucleotides in length. The PCR reaction is performed with an equimolar mix of eight distinct oligonucleotide primers: four forward and four reverse oligonucleotide primer species. For example, the forward primers 201 may include phase-shift regions 205 of 6, 7, 8, and 9 nucleotides in length. The four reverse primers 202 may also include phase-shift regions 211 of 6, 7, 8, and 9 nucleotides in length. The four species of forward primer 201 are identical to each other except for the phase-shift region 205, and the four species of reverse primers 202 are identical to each other except for the phase-shift region 211. Because of the equimolar mix of primer species in the PCR reaction of FIG. 2A, the products of the PCR reaction will be of incrementally varying lengths according to the particular SP1 primer pairs 201, 202 that amplified any particular amplicon.

The indexing sequences (i.e. sample tag or barcode) 204, 212 are unique user-defined identifying sequences that may be used to identify and distinguish samples during pooled SBS. Unique indexing sequences are assigned to each sample in the pool and resulting reads can be sorted and assigned to the sample from which they were read. Indexing sequences may be between 5 and 50 nucleotides in length.

The P5 and P7 sequences 203, 213 are located at the 5′ terminal ends of the oligonucleotide primers 201, 202. The P5 sequence 203 typically is located at the 5′ terminal end of the forward PCR primer 201 and the P7 sequence 213 typically is located at the 5′ terminal end of the reverse PCR primer 202. The P5 and P7 sequences 203, 213 allow the library fragment 222 to attach to a flow cell surface by annealing to complementary oligos immobilized to the glass. Before attachment to the flow cell, the target DNA fragments 222 are denatured, and thus a single-stranded copy of the target DNA is sequenced.

The amplicon products 222 of the PCR reaction of FIG. 2A, illustrated in FIG. 2B, include the following features: P5 sequence 214, indexing sequence (i.e. sample tag or barcode) 215, phase-shift region 216, target DNA binding sites 217, target DNA binding site 218, phase-shift region 219, indexing sequence (i.e. sample tag or barcode) 220, and P7 sequence 221. Amplicon products 222 may be purified and used as input material for downstream SBS applications. Phase-shift regions 216, 219 introduce nucleotide diversity to what would otherwise have been homogenous DNA ends with identical or nearly identical nucleotide sequence.

In yet another embodiment—a “four-oligo, one-step” embodiment—referring again to FIG. 1, a single PCR reaction may be performed using the four oligonucleotide primers 101, 102, 112, and 113. Forward primer 101 and reverse primer 102 target and amplify DNA fragment 107, yielding PCR product 120 . As product 120 accumulates in the reaction mixture, it is targeted and amplified by forward primer 112 and reverse primer 113, yielding PCR product 137.

The final amplicon product 137 of the single PCR reaction mediated by primers 101, 102, 112, and 113, include the cumulative features of the SP1 reaction of FIG. 1A and the SP2 reaction of FIG. 1B, namely: P5 sequence 127, indexing sequence (i.e. sample tag or barcode) 128, SP1 amplicon target binding sites 129, phase-shift region 130, target DNA binding sites 131, target DNA binding site 132, phase-shift region 133, SP2 primer binding site 134, indexing sequence (i.e. sample tag or barcode) 135, and P7 sequence 136. Amplicon products 137 may be purified and used as input material for downstream SBS applications. Phase-shift regions 130, 133 introduce nucleotide diversity to what would otherwise have been homogenous DNA ends with identical or nearly identical nucleotide sequence.

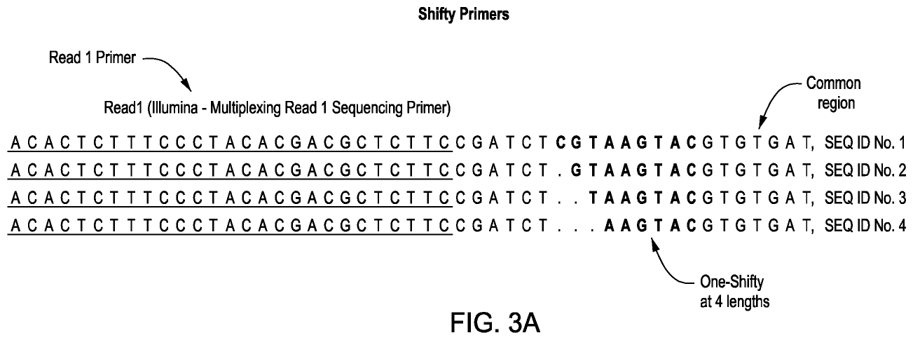

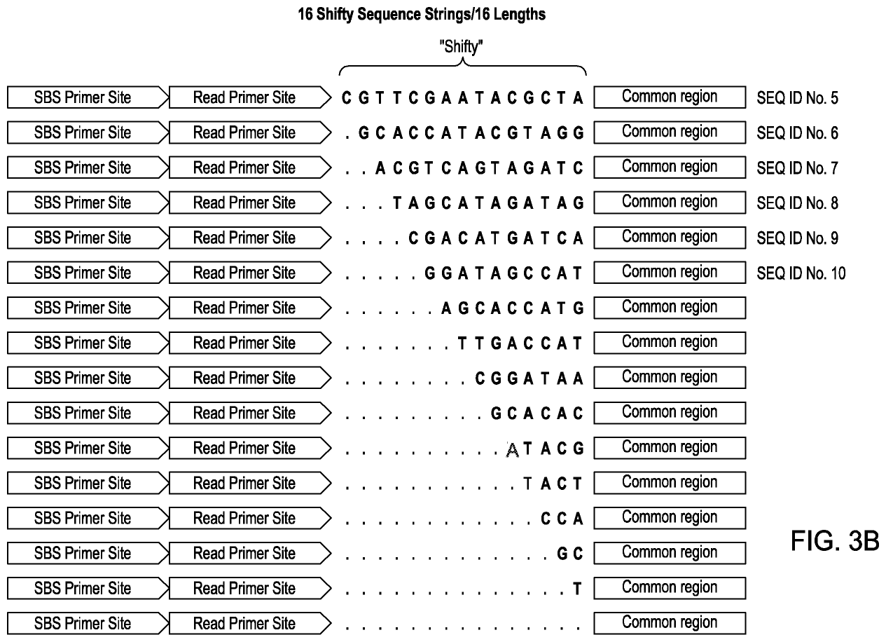

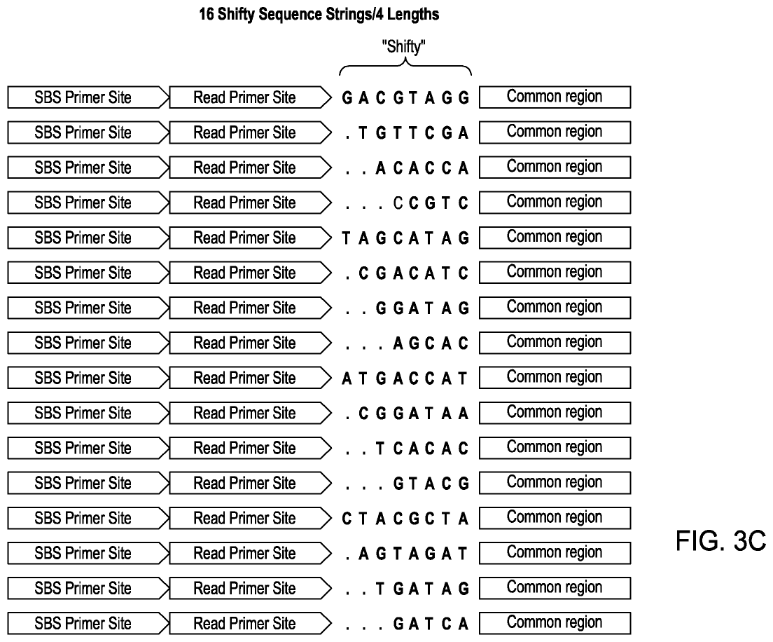

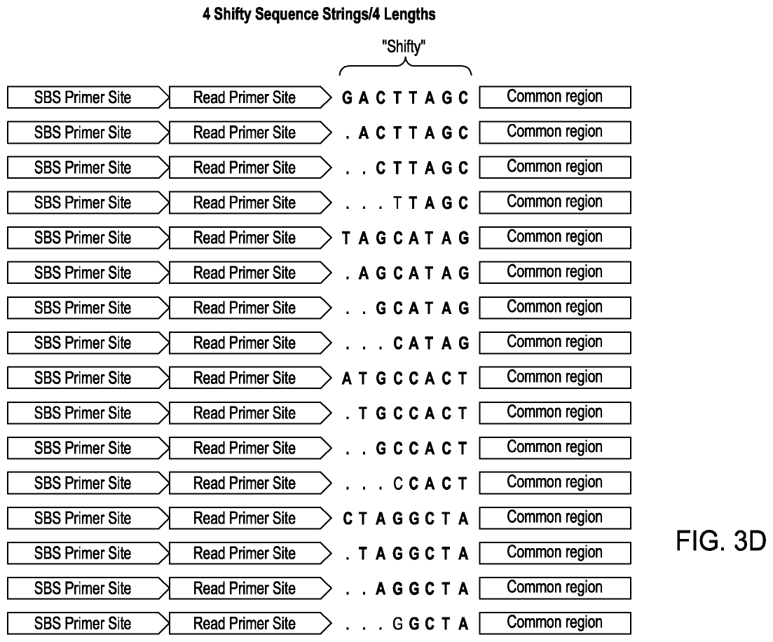

FIG. 3A shows the nucleotide sequences of an ILLUMINA™ sequencing primer in an exemplary set of four shifty forward primers (e.g., only the forward primers are shown in this FIG. 3A). In FIG. 3A, there is an ILLUMINA™ sequencing primer 5′ of a linker sequence “CGATCT”, which is 5′ of a “shifty” sequence. The first shifty sequence begins with a cytosine residue at the 5′ end; namely “CGTAAGTACGTGTGAT” [SEQ ID NO. 11; which is a portion of SEQ ID NO.1], where the sequence common to all shifty primers is “AGTACGTGTGAT” [SEQ ID NO. 12]. The second shifty sequence begins with a guanine residue at the 5′ end; namely “GTCAAGTACGTGTGAT” [SEQ ID NO. 13; which is a portion of SEQ ID NO.1], again containing the common sequence “AGTACGTGTGAT” [SEQ ID NO. 12]. The third shift sequence begins with a thymine residue at the 5′ end; namely “TAAGTACGTGTGAT” [SEQ ID NO. 14; which is a portion of SEQ ID NO.1], again containing the common sequence “AGTACGTGTGAT” [SEQ ID NO. 12]. The fourth shifty sequence begins with an adenine residue at the 5′ end; namely “AAGTACGTGTGAT” [SEQ ID NO. 15; which also is a portion of SEQ ID NO.1], again containing the common sequence “AGTACGTGTGAT” [SEQ ID NO. 12]. A “.” represents a missing nucleotide; thus the second shifty sequence is one nucleotide shorter than the first shifty sequence, the third shifty sequence is one nucleotide shorter than the second shifty sequence and two nucleotides shorter than the first shifty sequence, and the fourth shifty sequence is one nucleotide shorter than the third shifty sequence, two nucleotides shorter than the second shifty sequence and three nucleotides shorter than the first shifty sequence. Each nucleotide A, T, G and C is represented as a 5′ residue for training purposes. In this example, there are four shifty sequences, all with the same common sequence, for a set of four shifty sequences and four lengths. As described above, the shifty sequences are but one portion or component of a shifty primer.

FIG. 3B shows an alternative exemplary set of forward shifty primers where the set of forward primers comprises sixteen different shifty sequences and sixteen different shifty sequence lengths with each nucleotide A, T, G and C represented as a 5′ residue four times for training purposes. In this case, each forward shifty primer comprises an SBS primer site, a read primer site (e.g., ILLUMINA™ sequencing primer site), a shifty sequence and a common region 3′ of the shifty sequence. The first shifty sequence begins with a cytosine residue at the 5′ end, namely “CGTTCGAATACGCTA” [SEQ ID NO. 5]; the second shifty sequence begins with a guanine at the 5′ end, namely “GCACCATACGTAGG” [SEQ ID NO. 6]; the third shifty sequence begins with an adenine residue at the 5′ end, namely “ACGTCAGTAGATC” [SEQ ID NO. 7]; the fourth shifty sequence begins with a thymine residue at the 5′ end, namely “TAGCATAGATAG” [SEQ ID NO. 8] and so on. In this embodiment there are 16 different shifty sequences and lengths for the forward primer set, all having a common region; however, it should be noted that there could be 8 different sequences and lengths, 8 different lengths where some nucleotides or all nucleotides except the 5′ nucleotide are the same; 12 different sequences and lengths, 12 different lengths where some nucleotides or all nucleotides except the 5′ nucleotide are the same; 20 different sequences and lengths, 20 different lengths where some nucleotides or all nucleotides except the 5′ nucleotide are the same. That is, the sets of shifty sequences must comprise at least four shifty sequences where each nucleotide A, T, G and C is represented as a 5′ residue for training purposes for the forward primers and for the reverse primers; however, there may be more than four shifty sequences, but preferably there are multiples of four shifty sequences so that each nucleotide A, T, G and C is represented as a 5′ residue on an equal number of shifty sequences for training purposes. Again, only the forward primers are shown in this FIG. 3B; thus, the set of shifty oligo primers would be twice 16 or 32. Note also that in this particular example, the common region preferably has an adenine at its 5′ end to provide the fourth adenine for training.

FIG. 3C shows yet another exemplary set of shifty sequences where the set comprises sixteen sequence strings having four different lengths. Only the shifty sequences for the forward primers are shown. The first shifty sequence begins with a guanine residue at the 5′ end, namely “GACGTAGG” and is eight nucleotides in length; the second shifty sequence begins with a thymine at the 5′ end, namely “TGTTCGA” and is seven nucleotides in length; the third shifty sequence begins with an adenine residue at the 5′ end, namely “ACACCA” and is six nucleotides in length; the fourth shifty sequence begins with a cytosine residue at the 5′ end, namely “CCGTC” and is five nucleotides in length and so on. Note that each shifty sequence in the set of sixteen shifty sequences has a common region; however the shifty portion of the shifty sequences are all different. Also—in contrast to FIG. 3B—instead of the sixteen shifty sequences being sixteen different lengths, the sixteen shifty sequences are one of four different lengths (e.g., five to eight nucleotides in length) with each nucleotide A, T, G and C is represented as a 5′ residue at each length.

FIG. 3D shows an exemplary set of sixteen forward shifty primers where the set comprises four different shifty sequence motifs and four lengths (again, only forward primers are shown). The first shifty sequence begins with a guanine residue at the 5′ end, namely “GACGTAGC”; the second shifty sequence begins with an adenine at the 5′ end, namely “ACGTAGC”; the third shifty sequence begins with a cytosine residue at the 5′ end, namely “CGTAGC”; the fourth shifty sequence begins with a thymine residue at the 5′ end, namely “TTAGC” and so on. Note that each shifty sequence in the set of sixteen shifty sequences (e.g., shifty forward primers) has a common region and the shifty portion of the shifty sequences are the same for a four-member set of ATCG shifty sequences. That is, there are four shifty sequence motifs. One member of each set is 8 nucleotides in length, one member is 7 nucleotides in length, one member is 6 nucleotides in length and one member is 5 nucleotides in length.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Other equivalent methods, steps and compositions are intended to be included in the scope of the invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1: Introducing Nucleotide Diversity to Homogenous DNA Ends by Successive PCR Reactions During Sample Preparation for Sequencing by Synthesis (SBS)

### Capture Primer Stock:

SP1 primers were ordered and commercially synthesized by Integrated DNA Technologies, Inc. (Coralville, Iowa). Four “forward” and four “reverse” oligonucleotide primers were ordered, each containing identical target binding sequences and identical SP2 primer binding sequences, with four classes of phase-shift regions of varying length, for example, 6, 7 8, or 9 nucleotides. Each oligonucleotide primer was ordered at a concentration of 50 μM. The eight unique oligonucleotide primers, i.e. four forward each with a phase-shift region of unique length and four reverse each with a phase-shift region of unique length, were pooled in the following proportions to make a 10 μM Capture Primer Stock (CPS): 10 μL each of eight unique oligonucleotide primers at a stock concentration of 50 μM and 320 μL of 1×TE buffer.

### Sample for Sequencing:

DNA target fragments for sequencing were purified and concentrated by using the AMPure® PCR purification kit (Beckman Coulter Life Science, Indianapolis, Ind.). 5 μL of eluate was diluted in 45 μL of 1×TE buffer to yield 50 μL of ten-fold diluted target sample for sequencing.

### Sequencing PCR Amplification (SPA) Mix:

A PCR mix excluding oligonucleotide primers and target DNA was prepared in the following proportions: 1425 μL PCR-grade water; 500 μL 5× Phusion HF buffer (Thermo Fisher Scientific, Waltham, Mass.); 50 μL dNTPs at a concentration of 10 mM (New England Biolabs, Ipswich, Mass.); 25 μL Phusion Hot Start II DNA Polymerase at a concentration of 2 U/μL (Thermo Fisher Scientific, Waltham, Mass.). 2000 μL of this SPA mix was sufficient for 50 individual 50 μL SP1 reactions.

### Sequencing PCR Reaction #1 (SP1):

The first sequencing PCR reaction, to amplify target DNA and introduce phase-shift sequences and SP2 primer binding sites, was prepared as follows for a single reaction: 40 μL sequencing PCR amplification (SPA) mix; 5 μL capture primer stock (CPS); 5 μL ten-fold diluted target DNA. The 40 μL SPA mix was aliquoted into each well, CPS stock was added, then ten-fold diluted target DNA was added. The 50 μL reaction was thermocycled according to the following program: (step #1) 2 minutes at 98° C.; (step #2) 10 seconds at 98° C.; (step #3) 2 minutes 30 seconds at 72° C.; (step #4) cyclically repeat step #2 and step #3 seven additional times; (step #5) 5 minutes at 72° C.; (step #6) hold at 4° C. indefinitely.

The resulting amplicons from SP1 reactions were 81 to 87 basepairs longer than the target captured sequence, resulting from the addition of the varying phase-shift sequences and the SP2 primer-binding sites. 5 μL of the product of each SP1 reaction was diluted in 495 μL of PCR-grade water to yield 100-fold diluted SP1 reaction products.

### Index/SampleTag Primer Stock (IPS):

SP2 primers were ordered and commercially synthesized by Integrated DNA Technologies, Inc. (Coralville, Iowa). One SP2 “forward” and one SP2 “reverse” oligonucleotide primers were ordered, each containing unique indexing or “barcode” sequences specific to each target sample, and P5 or P7 sequences (Illumina, Inc., San Diego, Calif.). Each oligonucleotide primer was ordered at a concentration of 50 μM. An index primer stock (IPS) mix was made by combining 10 μL of SP2 forward primer, 10 μL of SP2 reverse primer, and 80 μL of 1×TE buffer.

### Sequencing PCR Reaction #2 (SP2):

The second sequencing PCR reaction, to introduce indexing sequences and P5 or P7 sequences to fragment ends, was prepared as follows for a single reaction: 40 μL sequencing PCR amplification (SPA) mix; 5 μL indexing primer stock (IPS); 5 μL 100-fold diluted SP1 reaction products. The 40 μL SPA mix was aliquoted into each well, IPS stock was added, then 100-fold diluted SP1 reaction products were added. The 50 μL reactions were thermocycled according to the following program: (step #1) 2 minutes at 98° C.; (step #2) 10 seconds at 98° C.; (step #3) 2 minutes 30 seconds at 72° C.; (step #4) cyclically repeat step #2 and step #3 nine additional times; (step #5) 5 minutes at 72° C.; (step #6) hold at 4° C. indefinitely.

The resulting amplicons from SP2 reactions were 71 basepairs longer than the SP1 reaction products, resulting from the addition of the indexing sequences and P5 or P7 sequences. SP2 reaction products were purified using the AMPure® PCR purification kit (Beckman Coulter Life Science, Indianapolis, Ind.) and were then ready for SBS on a flow cell as described above.

### Example 2: Introducing Nucleotide Diversity to Homogenous DNA Ends by a Single PCR Reaction During Sample Preparation for Sequencing by Synthesis (SBS)

### Capture Primer Stock:

Oligonucleotide primers were ordered and commercially synthesized by Integrated DNA Technologies, Inc. (Coralville, Iowa). Four “forward” and four “reverse” oligonucleotide primers were ordered, each containing identical target binding sequences, but with four classes of phase-shift regions of varying length, for example, 6, 7 8, or 9 nucleotides. Each oligonucleotide primer was ordered at a concentration of 50 μM. The eight unique oligonucleotide primers, i.e. four forward each with a phase-shift region of unique length and four reverse each with a phase-shift region of unique length, were pooled in the following proportions to make a 10 μM Capture Primer Stock (CPS): 10 μL each of eight unique oligonucleotide primers at a stock concentration of 50 μM and 320 μL of 1×TE buffer.

### Sample for Sequencing:

DNA target fragments for sequencing were purified and concentrated by using the AMPure® PCR purification kit (Beckman Coulter Life Science, Indianapolis, Ind.). 5 μL of eluate was diluted in 45 μL of 1×TE buffer to yield 50 μL of ten-fold diluted target sample for sequencing.

### Sequencing PCR Amplification (SPA) Mix:

A PCR mix excluding oligonucleotide primers and target DNA was prepared in the following proportions: 1425 μL PCR-grade water; 500 μL 5× Phusion HF buffer (Thermo Fisher Scientific, Waltham, Mass.); 50 μL dNTPs at a concentration of 10 mM (New England Biolabs, Ipswich, Mass.); 25 μL Phusion Hot Start II DNA Polymerase at a concentration of 2 U/μL (Thermo Fisher Scientific, Waltham, Mass.). 2000 μL of this SPA mix was sufficient for 50 individual 50 μL SP1 reactions.

### Library Preparation PCR Reaction:

The library preparation PCR reaction, to amplify target DNA and introduce phase-shift sequences, indexing sequences, and P5 and P7 sequences (Illumina, Inc., San Diego, Calif.), was prepared as follows for a single reaction: 40 μL sequencing PCR amplification (SPA) mix; 5 μL capture primer stock (CPS); 5 μL ten-fold diluted target DNA. The 40 μL SPA mix was aliquoted into each well, CPS stock was added, then ten-fold diluted target DNA was added. The 50 μL reaction was thermocycled according to the following program: (step #1) 2 minutes at 98° C.; (step #2) 10 seconds at 98° C.; (step #3) 2 minutes 30 seconds at 72° C.; (step #4) cyclically repeat step #2 and step #3 seven additional times; (step #5) 5 minutes at 72° C.; (step #6) hold at 4° C. indefinitely.

The resulting amplicons from the library preparation PCR reactions were 152 to 158 basepairs longer than the target captured sequence, resulting from the addition of the varying phase-shift sequences, the indexing sequences, and the P5 and P7 sequences. Library preparation PCR reaction products were purified using the AMPure® PCR purification kit (Beckman Coulter Life Science, Indianapolis, Ind.) and were then ready for SBS on a flow cell as described above.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

I claim:

1. A set of oligonucleotides configured to introduce nucleotide diversity into sample DNA to be sequenced comprising:
a first primer comprising from 5′ to 3′, a first SP2 binding site, a first variable-length phase-shift sequence n nucleotides in length, and a region homologous to a 5′ region of the sample DNA;
a second primer comprising from 5′ to 3′, the first SP2 binding site, a second variable-length phase-shift sequence n+1 nucleotides in length, and the region homologous to a 5′ region of the sample DNA;
a third primer comprising from 5′ to 3′, the first SP2 binding site, a third variable-length phase-shift sequence n+2 nucleotides in length, and the region homologous to a 5′ region of the sample DNA;
a fourth primer comprising from 5′ to 3′, the first SP2 binding site, a fourth variable-length phase-shift sequence n+3 nucleotides in length, and the region homologous to a 5′ region the sample DNA;
a fifth primer comprising from 5′ to 3′, a second SP2 binding site, a fifth variable-length phase-shift sequence n nucleotides in length, and a region homologous to a 3′ region of the sample DNA;
a sixth primer comprising from 5′ to 3′, the second SP2 binding site, a sixth variable-length phase-shift sequence n+1 nucleotides in length, and the region homologous to a 3′ region of the sample DNA;
a seventh primer comprising from 5′ to 3′ the second SP2 binding site, a seventh variable-length phase-shift sequence n+2 nucleotides in length, and the region homologous to a 3′ region of the sample DNA; and
an eighth primer comprising from 5′ to 3′, the second SP2 binding site, an eighth variable-length phase-shift sequence n+3 nucleotides in length, and the region homologous to a 3′ region of the sample DNA; wherein the first, second, third and fourth primers have different nucleotides at a 5′ position of their variable-length phase-shift sequences, and wherein the fifth, sixth, seventh and eighth primers have different nucleotides at a 5′ position of their variable-length phase-shift sequences.

2. The set of oligonucleotides of claim 1, wherein, except for the 5′ position, the first, second, third, fourth, fifth, sixth, seventh and eighth variable-length phase-shift sequences are a same sequence of nucleotides.

3. The set of oligonucleotides of claim 1, wherein the first, second, third, fourth, fifth, sixth, seventh and eighth variable-length phase-shift sequences are different sequences.

4. The set of oligonucleotides of claim 1, wherein the first, second, third and fourth primers further comprise: a first SP1 binding site configured to hybridize to the first SP2 binding site, an indexing sequence, and a P5 sequence if fifth, sixth, seventh, and eighth primers comprise a P7 sequence and the P7 sequence if the fifth, sixth, seventh, and eighth primers comprise the P5 sequence; and wherein the fifth, sixth, seventh, and eighth primers further comprise: a second SP1 binding site configured to hybridize to the second SP2 binding site, the indexing sequence, and the P7 sequence if the first, second, third and fourth primers comprise the P5 sequence and the P5 sequence if the first, second, third and fourth primers comprise the P7 sequence.

5. The set of oligonucleotides of claim 2, wherein the P5 sequence is 5′-AATGATACGGCGACCACCCA-3′ [SEQ ID NO. 16] and the P7 sequence is 5′-CAAGCAGAAGACGGCATACGAGAT-3′ [SEQ ID NO. 17].

6. The set of oligonucleotides of claim 1 further comprising a ninth primer comprising: a first SP1 binding site configured to hybridize to the first SP2 binding site, an indexing sequence, and a P5 sequence if a tenth primer comprises a P7 sequence and the P7 sequence if the tenth primer comprises the P5 sequence; and the tenth primer comprising: a second SP1 binding site configured to hybridize to the second SP2 binding site, the indexing sequence, and the P7 sequence if the ninth primer comprises the P5 sequence and the P5 sequence if the ninth primer comprises the P7 sequence.

7. The set of oligonucleotides of claim 4, wherein the P5 sequence is 5′-AATGATACGGCGACCACCCA-3′ [SEQ ID NO. 16] and the P7 sequence is 5′-CAAGCAGAAGACGGCATACGAGAT-3′ [SEQ ID NO. 17].

8. The set of oligonucleotides of claim 1, wherein the first, second, third, fourth, fifth, sixth, seventh, and eighth primers are represented at about equimolar ratios.

9. The set of oligonucleotides of claim 1, wherein the first, second, third, fourth, fifth, sixth, seventh, and eighth variable-length phase-shift sequences have a sequence motif A.

10. The set of oligonucleotides of claim 1, further comprising:
a ninth primer comprising from 5′ to 3′, the first SP2 binding site, a ninth variable-length phase-shift sequence n nucleotides in length, and the region homologous to a 5′ region of the sample DNA;
a tenth primer comprising from 5′ to 3′, the first SP2 binding site, a tenth variable-length phase-shift sequence n+1 nucleotides in length, and the region homologous to a 5′ region of the sample DNA;
an eleventh primer comprising from 5′ to 3′, the first SP2 binding site, an eleventh variable-length phase-shift sequence n+2 nucleotides in length, and the region homologous to a 5′ region of the sample DNA;
a twelfth primer comprising from 5′ to 3′, the first SP2 binding site, a twelfth variable-length phase-shift sequence n+3 nucleotides in length, and the region homologous to a 5′ region the sample DNA;
a thirteenth primer comprising from 5′ to 3′, a second SP2 binding site, a thirteenth variable-length phase-shift sequence n nucleotides in length, and a region homologous to a 3′ region of the sample DNA;
a fourteenth primer comprising from 5′ to 3′, the second SP2 binding site, a fourteenth variable-length phase-shift sequence n+1 nucleotides in length, and the region homologous to a 3′ region of the sample DNA;
a fifteenth primer comprising from 5′ to 3′ the second SP2 binding site, a fifteenth variable-length phase-shift sequence n+2 nucleotides in length, and the region homologous to a 3′ region of the sample DNA; and
a sixteenth primer comprising from 5′ to 3′, the second SP2 binding site, a sixteenth variable-length phase-shift sequence n+3 nucleotides in length, and the region homologous to a 3′ region of the sample DNA; wherein the ninth, tenth, eleventh and twelfth primers have different nucleotides at a 5′ position of their variable-length phase-shift sequences, wherein the thirteenth, fourteenth, fifteenth and sixteenth primers have different nucleotides at a 5′ position of their variable-length phase-shift sequences, and wherein the variable-length phase-shift sequence of each of the ninth, tenth, eleventh and twelfth primers is a different sequence than the variable-length phase-shift sequence of the first, second, third, fourth, fifth, sixth, seventh and eighth primers.

11. The set of oligonucleotides of claim 10, wherein the ninth, tenth, eleventh and twelfth variable-length phase-shift sequences are different sequences than the thirteenth, fourteenth, fifteenth and sixteenth variable-length phase-shift sequences.

12. The set of oligonucleotides of claim 10, wherein the first, second, third and fourth primers further comprise: a first SP1 binding site configured to hybridize to the first SP2 binding site, an indexing sequence, and a P5 sequence if the fifth, sixth, seventh, and eighth primers comprise a P7 sequence and the P7 sequence if the fifth, sixth, seventh, and eighth primers comprise the P5 sequence; wherein the fifth, sixth, seventh, and eighth primers further comprise: a second SP1 binding site configured to hybridize to the second SP2 binding site, the indexing sequence, and the P7 sequence if the first, second, third and fourth primers comprise the P5 sequence and the P5 sequence if the first, second, third and fourth primers comprise the P7 sequence; the ninth, tenth, eleventh and twelfth primers further comprise: the first SP1 binding site configured to hybridize to the first SP2 binding site, the indexing sequence; and the P5 sequence if the thirteenth, fourteenth, fifteenth and sixteenth primers comprise the P7 sequence and the P7 sequence if the thirteenth, fourteenth, fifteenth and sixteenth primers comprise the P5 sequence; and the thirteenth, fourteenth, fifteenth and sixteenth primers further comprise: the second SP1 binding site configured to hybridize to the second SP2 binding site, the indexing sequence, and the P7 sequence if the ninth, tenth, eleventh and twelfth primers comprise the P5 sequence and the P5 sequence if the ninth, tenth, eleventh and twelfth primers comprise the P7 sequence.

13. The set of oligonucleotides of claim 10, wherein, except for the 5′ position, the first, second, third, fourth, fifth, sixth, seventh and eight variable-length phase-shift sequences have a sequence motif A and the ninth, tenth, eleventh, twelfth, thirteenth, fourteenth, fifteenth and sixteenth variable-length phase-shift sequences have a sequence motif B, and wherein sequence motif A and sequence motif B are different.

14. The set of oligonucleotides of claim 10, wherein the P5 sequence is 5′-AATGATACGGCGACCACCCA-3′ [SEQ ID NO. 16] and the P7 sequence is 5′-CAAGCAGAAGACGGCATACGAGAT-3′ [SEQ ID NO. 17].

15. The set of oligonucleotides of claim 10 further comprising a seventeenth primer comprising: a first SP1 binding site configured to hybridize to the first SP2 binding site, an indexing sequence, and a P5 sequence if a tenth primer comprises a P7 sequence and the P7 sequence if the tenth primer comprises the P5 sequence; and an eighteenth primer comprising: a second SP1 binding site configured to hybridize to the second SP2 binding site, the indexing sequence, and the P7 sequence if the ninth primer comprises the P5 sequence and the P5 sequence if the ninth primer comprises the P7 sequence.

16. The set of oligonucleotides of claim 10, further comprising:
a seventeenth primer comprising from 5′ to 3′, the first SP2 binding site, a seventeenth variable-length phase-shift sequence n nucleotides in length, and the region homologous to a 5′ region of the sample DNA;
an eighteenth primer comprising from 5′ to 3′, the first SP2 binding site, an eighteenth variable-length phase-shift sequence n+1 nucleotides in length, and the region homologous to a 5′ region of the sample DNA;
a nineteenth primer comprising from 5′ to 3′, the first SP2 binding site, a nineteenth variable-length phase-shift sequence n+2 nucleotides in length, and the region homologous to a 5′ region of the sample DNA;
a twentieth primer comprising from 5′ to 3′, the first SP2 binding site, a twentieth variable-length phase-shift sequence n+3 nucleotides in length, and the region homologous to a 5′ region the sample DNA;
a twenty-first primer comprising from 5′ to 3′, a second SP2 binding site, a twenty-first variable-length phase-shift sequence n nucleotides in length, and a region homologous to a 3′ region of the sample DNA;
a twenty-second primer comprising from 5′ to 3′, the second SP2 binding site, a twenty-second variable-length phase-shift sequence n+1 nucleotides in length, and the region homologous to a 3′ region of the sample DNA;
a twenty-third primer comprising from 5′ to 3′ the second SP2 binding site, a twenty-third variable-length phase-shift sequence n+2 nucleotides in length, and the region homologous to a 3′ region of the sample DNA; and
a twenty-fourth primer comprising from 5′ to 3′, the second SP2 binding site, a twenty-fourth variable-length phase-shift sequence n+3 nucleotides in length, and the region homologous to a 3′ region of the sample DNA; wherein the seventeenth, eighteenth, nineteenth and twentieth primers have different nucleotides at a 5′ position of their variable-length phase-shift sequences, wherein the twenty-first, twenty-second, twenty-third and twenty-fourth primers have different nucleotides at a 5′ position of their variable-length phase-shift sequences, and wherein the seventeenth, eighteenth, nineteenth, twentieth, twenty-first, twenty-second, twenty-third and twenty-fourth variable-length phase-shift sequences are different sequences than the first, second, third, fourth, fifth, sixth, seventh, eighth, ninth, tenth, eleventh, twelfth, thirteenth, fourteenth, fifteenth and sixteenth variable-length phase-shift sequences.

17. The set of oligonucleotides of claim 16, wherein, except for the 5′ position, the first, second, third, fourth, fifth, sixth, seventh and eighth variable-length phase-shift sequences have a sequence motif A, the ninth, tenth, eleventh, twelfth, thirteenth, fourteenth, fifteenth and sixteenth variable-length phase-shift sequences have a sequence motif B, and the seventeenth, eighteenth, nineteenth, twentieth, twenty-first, twenty-second, twenty-third and twenty-fourth variable-length phase-shift sequences have a sequence motif C, and wherein sequence motif A, sequence motif B and sequence motif C are different.

18. A set of oligonucleotides configured to introduce nucleotide diversity into sample DNA to be sequenced comprising:
a first primer comprising from 5′ to 3′, a P5 or P7 sequence, an indexing sequence, a first SP2 binding site, a first variable-length phase-shift sequence n nucleotides in length, and a region homologous to a 5′ region of the sample DNA;
a second primer comprising from 5′ to 3′, the P5 or P7 sequence, the indexing sequence, the first SP2 binding site, a second variable-length phase-shift sequence n+1 nucleotides in length, and the region homologous to a 5′ region of the sample DNA;
a third primer comprising from 5′ to 3′, the P5 or P7 sequence, the indexing sequence, the first SP2 binding site, a third variable-length phase-shift sequence n+2 nucleotides in length, and the region homologous to a 5′ region of the sample DNA;
a fourth primer comprising from 5′ to 3′, the P5 or P7 sequence, the indexing sequence, the first SP2 binding site, a fourth variable-length phase-shift sequence n+3 nucleotides in length, and the region homologous to a 5′ region the sample DNA;
a fifth primer comprising from 5′ to 3′, the P5 or P7 sequence, the indexing sequence, a second SP2 binding site; a fifth variable-length phase-shift sequence n nucleotides in length, and a region homologous to a 3′ region of the sample DNA;
a sixth primer comprising from 5′ to 3′, the P5 or P7 sequence, the indexing sequence, the second SP2 binding site, a sixth variable-length phase-shift sequence n+1 nucleotides in length, and the region homologous to a 3′ region of the sample DNA;
a seventh primer comprising from 5′ to 3′, the P5 or P7 sequence, the indexing sequence, the second SP2 binding site, a seventh variable-length phase-shift sequence n+2 nucleotides, and the region homologous to a 3′ region of the sample DNA; and
an eighth primer comprising from 5′ to 3′, the P5 or P7 sequence, the indexing sequence, the second SP2 binding site, an eighth variable-length phase-shift sequence n+3 nucleotides in length, and the region homologous to a 3′ region of the sample DNA;
wherein the first, second, third and fourth primers have different nucleotides at a 5′ position of their variable-length phase-shift sequences, and wherein the fifth, sixth, seventh and eighth primers have different nucleotides at a 5′ position of their variable-length phase-shift sequences and wherein if the first, second, third and fourth primers comprise P5 sequences, the sixth, seventh, eighth and ninth primers comprise P7 sequences and wherein if the first, second, third and fourth primers comprise P7 sequences, the sixth, seventh, eighth and ninth primers comprise P5 sequences.

19. The set of oligonucleotides of claim 18, wherein, except for the 5′ position, the first, second, third, fourth, fifth, sixth, seventh and eighth variable-length phase-shift sequences are a same sequence of nucleotides.

20. The set of oligonucleotides of claim 18, wherein the first, second, third, fourth, fifth, sixth, seventh and eighth variable-length phase-shift sequences are different sequences.


SEQUENCE LISTING (17 sequences)

SEQ ID NO: 1 - DNA, 48 bp, Artificial Sequence
  SHIFTY SEQUENCE
acactctttccctacacgacgctcttcgatctcgtaagtacgtgtgat

SEQ ID NO: 2 - DNA, 47 bp, Artificial Sequence
  SHIFTY SEQUENCE
acactctttccctacacgacgctcttcgatctgtaagtacgtgtgat

SEQ ID NO: 3 - DNA, 46 bp, Artificial Sequence
  SHIFTY SEQUENCE
acactctttccctacacgacgctcttcgatcttaagtacgtgtgat

SEQ ID NO: 4 - DNA, 45 bp, Artificial Sequence
  SHIFTY SEQUENCE
acactctttccctacacgacgctcttcgatctaagtacgtgtgat

SEQ ID NO: 5 - DNA, 15 bp, Artificial Sequence
  SHIFTY SEQUENCE
cgttcgaatacgcta

SEQ ID NO: 6 - DNA, 14 bp, Artificial Sequence
  SHIFTY SEQUENCE
gcaccatacgtagg

SEQ ID NO: 7 - DNA, 13 bp, Artificial Sequence
  SHIFTY SEQUENCE
acgtcagtagatc

SEQ ID NO: 8 - DNA, 12 bp, Artificial Sequence
  SHIFTY SEQUENCE
tagcatagatag

SEQ ID NO: 9 - DNA, 11 bp, Artificial Sequence
  SHIFTY SEQUENCE
cgacatgatca

SEQ ID NO: 10 - DNA, 10 bp, Artificial Sequence
  SHIFTY SEQENCE
ggatagccat

SEQ ID NO: 11 - DNA, 16 bp, Artificial Sequence
  SHIFTY SEQUENCE
cgtaagtacgtgtgat

SEQ ID NO: 12 - DNA, 12 bp, Artificial Sequence
  SHIFTY SEQUENCE
agtacgtgtgat

SEQ ID NO: 13 - DNA, 16 bp, Artificial Sequence
  SHIFTY SEQUENCE
gtcaagtacgtgtgat

SEQ ID NO: 14 - DNA, 14 bp, Artificial Sequence
  SHIFTY SEQUENCE
taagtacgtgtgat

SEQ ID NO: 15 - DNA, 13 bp, Artificial Sequence
  SHIFTY SEQUENCE
aagtacgtgtgat

SEQ ID NO: 16 - DNA, 20 bp, Artificial Sequence
  SEQUENCING PRIMER
aatgatacggcgaccaccca

SEQ ID NO: 17 - DNA, 24 bp, Artificial Sequence
  SEQUENCING PRIMER
caagcagaagacggcatacgagat